(19) United States
(12) Patent Application Publication
(10) Pub. No.: US 2024/0417705 A1
(43) Pub. Date: Dec. 19, 2024

(54) NICKASE ENCODED BY SHEWANELLA INTEGRATED TEMPERATE PHAGE FRAGMENTS AND ITS APPLICATION

(71) Applicant: GUANGDONG GENERAL HOSPITAL, Guangzhou (CN)

(72) Inventors: Xiaoxiao LIU, Guangzhou (CN); Bing GU, Guangzhou (CN); Yunhu ZHAO, Guangzhou (CN); Ni ZHANG, Guangzhou (CN); Yong LING, Guangzhou (CN); Kaixuan YUAN, Guangzhou (CN); Shanzhao CUI, Guangzhou (CN)

(73) Assignee: GUANGDONG GENERAL HOSPITAL, Guangzhou (CN)

(21) Appl. No.: 18/512,064

(22) Filed: Nov. 17, 2023

(30) Foreign Application Priority Data
Jun. 19, 2023 (CN) 202310733318.X

(51) Int. Cl. C12N 9/16; C12N 1/20; C12N 15/52

(52) U.S. Cl. CPC C12N 9/16; C12N 1/205; C12N 15/52

(57) ABSTRACT

A nickase encoded by Shewanella integrated temperate phage fragments and an application thereof are provided. The nickase is derived from Shewanella oneidensis MR-1 (ATCC 700550), the amino acid sequence of the nickase is as shown in SEQ ID NO: 2, and the nucleotide sequence of the nickase is as shown in SEQ ID NO: 1. It can break a specific part of one of single strands of double-stranded DNA. The nickase has a wide range of action temperatures and good cutting effects, and has wide application values in the fields of nucleic acid isothermal amplification and the like.

### CROSS REFERENCE TO THE RELATED APPLICATIONS

This application is based upon and claims priority to Chinese Patent Application No. 202310733318.X, filed on Jun. 19, 2023, the entire contents of which are incorporated herein by reference

### SEQUENCE LISTING

The instant application contains a Sequence Listing which has been submitted in XML format via EFS-Web and is hereby incorporated by reference in its entirety. Said XML copy is named GBKY099_Sequence Listing.xml, created on Sep. 27, 2023, and is 3,966 bytes in size.

### TECHNICAL FIELD

The present invention relates to the field of biotechnology, in particular to a nickase encoded by Shewanella integrated temperate phage fragments and an application thereof.

### BACKGROUND

Nucleic acid detection is widely used in food safety, biomedical testing, environmental testing and other fields. A nucleic acid sequence-specific isothermal and polymerase chain reaction (PCR) amplification technology is a typical representative of the vigorous development of molecular diagnostics in recent years. Nickase (Nicking nuclease) can effectively replace DNA double-strand endonuclease for a rapid constant temperature amplification technology, which makes the isothermal amplification technology enter a new stage of development and move towards practical application faster. Strand displacement amplification (SDA) was proposed by Walker et al. in 1992 for the rapid detection of Mycobacterium tuberculosis IS6110 nucleic acid fragments. However, HincII cuts double strands of DNA. In order to allow it to cut only one of the double strands and obtain a gap to ensure that the subsequent strand displacement amplification reaction can proceed smoothly, a modified substrate (dATPoS) is introduced to form modified cutting sites to solve this problem. However, this leads to high cost and cumbersome procedure of this method, greatly limiting its wide application. The nickase (nicking nuclease) only cuts the single strand of nucleic acid, properly solves the problem of nucleic acid double-strand breaks generated by HincII, greatly simplifies the experimental steps, and reduces the possibility of sample contamination. Consequently, the method has stronger practical prospects for rapid and instant detection of nucleic acid on site (Point-Of-care testing, POCT). Genetic modification is one of the effective means to improve the activity and cutting efficiency of nickases (nicking nuclease). However, currently there are not enough nickase-producing bacteria that can be used for biological development, and the scope of genetic modification is limited. Therefore, it is of great application value to find producing bacteria that can produce nickases (nicking nuclease).

### SUMMARY

In view of this, the technical problem to be solved by the present invention is to provide a nickase encoded by Shewanella integrated temperate phage fragments and its application.

The present invention provides an application of Shewanella bacteria in the preparation of a nickase.

Further, the Shewanella bacteria is Shewanella oneidensis MR-1 (ATCC 700550) deposited in American Type Culture Collection (ATCC) with the accession number of ATCC No. 700550.

The present invention finds for the first time through digging that Shewanella oneidensis MR-1 (ATCC 700550) can produce a nickase (nicking nuclease), and further find through testing that the nickase (nicking nuclease) is encoded by a temperate phage fragment (SO_0641-SO_0690) integrated on a genome of Shewanella oneidensis MR-1 (ATCC 700550), in which an encoding gene SO_0655, which provides a new species source for nickase (nicking nuclease) donor bacteria and further enriches a nickase gene source.

The present invention provides a nickase derived from Shewanella oneidensis MR-1 (ATCC 700550).

Further, in the experiments of the present invention, it was found that a culture supernatant of Shewanella oneidensis MR-1 (ATCC 700550) functions as a nickase. After further verification, it was found that said Shewanella oneidensis MR-1 (ATCC 700550) can produce a new undisclosed nickase (nicking nuclease) whose amino acid sequence is shown in SEQ ID NO: 2.

Furthermore, the nickase (nicking nuclease) described in the present invention has a wide range of action temperatures (4 to 55° C.), not only can replace the existing DNA nickase (nicking nuclease), but also can be used in more experimental operations and technologies due to this wide range of action temperatures, thereby achieving more application values. At the same time, compared with other nickases (nicking nuclease), the nickase (nicking nuclease) according to the present invention has better cutting performance and/or enzyme activity, and also has a potential and space of further improvement in terms of effects, thereby providing a reliable gene source for genetic engineering transformation.

The present invention provides an application of a nickase in DNA single-strand break

Further, the DNA single-strand break includes the break of one of single strands in a double-stranded DNA, and also includes the direct break of a single-stranded DNA. In a specific embodiment of the present invention, the nickase can make one of the single strands of the double-stranded DNA break.

The present invention provides a nucleic acid encoding the nickase, which is encoded by a temperate phage (SO_0641-SO_0690) fragment integrated on the genome of Shewanella oneidensis MR-1 (ATCC 700550), and an encoding gene is SO_0655.

Further, a nucleotide sequence of the nucleic acid is shown in SEQ ID NO: 1, which is the first analysis of a function of a SO_0655 protein encoded by SEQ ID NO: 1.

The present invention provides a preparation method of a nickase, which is to cultivate Shewanella to obtain a culture containing the nickase; and

the Shewanella is Shewanella oneidensis MR-1 (ATCC 700550).

Further, the culture medium is LB, and a culture condition is 30° C., 200 rpm/min.

The present invention provides a preparation, which includes at least one of the nickase according to the present invention or a mixture containing the nickase prepared by the preparation method according to the present invention, and auxiliary materials.

In the present invention, the auxiliary materials are used to maintain the activity of the nickase or to make the nickase function better. In the present invention, the auxiliary materials include at least one of a stabilizer, an antioxidant, a reducing agent and/or a buffer.

The present invention provides a method for DNA single-strand break, which is to use the nickase or the preparation according to the present invention to cut DNA. Further, the cutting temperature is 4 to 55° C.

The present invention provides a new nickase, which is derived from Shewanella oneidensis MR-1 (ATCC 700550), an amino acid sequence of which is as shown in SEQ ID NO: 2, and a nucleotide sequence of which is as shown in SEQ ID NO: 1. It can break a specific part of one of the single strands of the double-stranded DNA. The nickase has a wide range of action temperatures and good cutting effects, and has wide application values in the fields of nucleic acid isothermal amplification and the like.

### BRIEF DESCRIPTION OF THE DRAWINGS

FIG. 1 shows an SDS-PAGE figure of a nickase, in which 100 mM 1 is an imidazole elution sample 1 of 100 mM, 100 mM 2 is an imidazole elution sample 2 of 100 mM; 300 mM 1 is an imidazole elution sample 1 of 300 mM, and 300 mM 2 is an imidazole elution sample 2 of 300 mM; 300 mM 3 is an imidazole sample 3 of 300 mM; and 10 mM, 20 mM, 30 mM, and 50 mM are respectively samples eluted by an imidazole of 10 mM, 20 mM, 30 mM, and 50 mM; and

FIG. 2 shows the verification of the nickase function, where OC is open circular DNA, L is linear DNA; and CCC is covalently closed circular DNA.

### DETAILED DESCRIPTION OF THE EMBODIMENTS

The present invention provides a nickase encoded by Shewanella integrated temperate phage fragments and its application. Those skilled in the art can learn from the content of the present invention and appropriately improve process parameters implementations. In particular, it should be pointed out that all similar replacements and modifications are obvious to those skilled in the art, and they are all considered to be included in the present invention. The method and application of the present invention have been described through preferred embodiments, and relevant personnel can obviously make changes or appropriate changes and combinations to the method and application herein without departing from the content, spirit and scope of the present invention to realize and apply the present invention technology.

A coding nucleotide sequence of the nickase is:

(SEQ ID NO: 1)
atggattttaatgcaatcgctaaggtaacgcaggattatgaggaagcct
gtgatcaaaaatcagtattggtcgactatctattaaaactgatcgatag
agctaatggcaatttaggcattgaacgggttaaacaagaggccataaaa
atcatggattgtgagttagccttccagcatcgacctgtgtttaatcccc
aaagcgatgattgctattgtgcattggatgtggtttttacaaagaacaa
agatgtagatatcatcattaacacattttatgtacataaatatggcttt
gtttttatcaaaaacgctgatgtatatgaaaactggttaactgatagag
aaatggattttaacagatgcgacgctaagagacaaagttttatccccta
tagtgtcagcgctacttaaacacttagaaaactaa;

An amino acid sequence of the nickase is:

(SEQ ID NO: 2)
MDFNAIAKVTQDYEEACDQKSVLVDYLLKLIDRANGNLGIERVKQEAIK
IMDCELAFQHRPVFNPQSDDCYCALDVVFTKNKDVDIIINTFYVHKYGF
VFIKNADVYENWLTDREMDLLTDATLRDKVLSPIVSALLKHLEN;

The test materials used in the present invention are all common commercially available products, which can be purchased in the market.

The present invention is further set forth below in conjunction with an embodiment:

Embodiment 1 Digging of nickase-producing bacteria and verification of nickase-producing bacteria

1. Cultivation of Shewanella and verification of nickase production

Shewanella oneidensis MR-1 (ATCC 700550) is a standard strain, which was found to produce nickases in experiments.

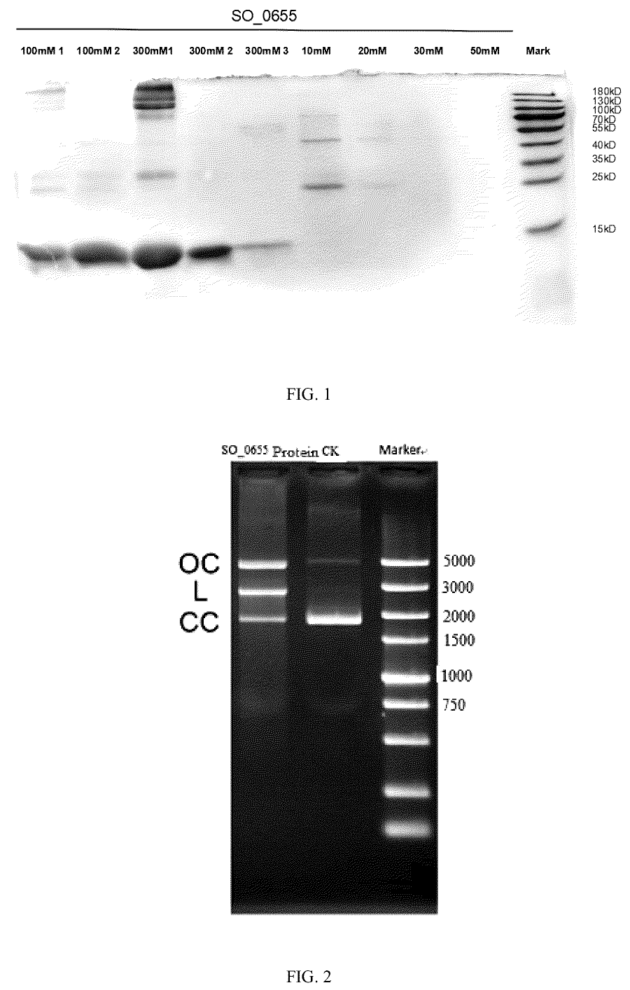

In the experiments, Shewanella oneidensis MR-1 (ATCC 700550) was fermented in an LB medium at 30° C. and 200 rpm/min. It was found that a supernatant of its lysate (PBS buffer solution) had the function of nuclease. After further test identification, it was found that the strain produces a new nickase, an amino acid sequence of which is shown in SEQ ID NO: 2. Further experiments and digging revealed that the nickase is encoded by a temperate phage (SO_0641-SO_0690) fragment integrated in a genome of Shewanella oneidensis MR-1, in which an encoding gene is SO_ 0655, and a nucleotide sequence is shown in SEQ ID NO: 1. Separately purifying the nickase is used to further verify the function of the nickase, and an SDS-PAGE figure of the nickase is shown in FIG. 1.

Verification of nickase function: Taking double-stranded DNA as an example, a reaction system contains: double-stranded DNA (0.6 μg) and SO_0655 purified protein, which, in 20 mM Tris-HCl buffer system, make up to 20 μL with water (SO_0655 protein content of 0.6 μg), under the condition of 4 to 55° C.; within 60 minutes, 0.3 pmol of double-stranded DNA was cut to produce single-strand break (as shown in FIG. 2), where OC is open circular DNA (single-stranded broken DNA), L is linear DNA (double-strand break); and CCC is covalently closed circular DNA (double-stranded circular DNA).

The above is only a preferred embodiment of the present invention, and it should be pointed out that for those of ordinary skill in the art, some improvements and modifications can also be made without departing from the principle of the present invention, and these improvements and modifications should also be considered as the protection scope of the present invention.

Sequence list

```
<?xml version=“1.0” encoding=“UTF-8”?>
<!DOCTYPE ST26SequenceListing PUBLIC “-//WIPO//DTD Sequence Listing 1.3//EN”
“ST26SequenceListing_V1_3.dtd″>
<ST26SequenceListing dtdVersion=“V1_3” fileName=“MP23008001.xml”
softwareName=“WIPO Sequence” software Version=“2.2.0” productionDate=“2023-06-19”>
              <ApplicantFileReference>MP23008001</ApplicantFileReference>
              <ApplicantName languageCode=“zh”> Guangdong Provincial People's Hospital
</ApplicantName>
              <ApplicantNameLatin>Guangdong Provincial People&apos;s
Hospital</ApplicantNameLatin>
              <InventionTitle languageCode=“zh”> Nickase encoded by Shewanella integrated
temperate phage fragments and its application </InventionTitle>
              <Sequence TotalQuantity>2</SequenceTotalQuantity>
              <SequenceData sequenceIDNumber=“1”>
                          <INSDSeq>
                                    <INSDSeq_length>429</INSDSeq_length>
                                    <INSDSeq_moltype>DNA</INSDSeq_moltype>
                                    <INSDSeq_division>PAT</INSDSeq_division>
                                    <INSDSeq_feature-table>
                                              <INSDFeature>
                                                        <INSDFeature_key>source</INSDFeature_key>
                                                        <INSDFeature_location>1 . . . 429</INSDFeature_location>
                                                        <INSDFeature_quals>
                                                                  <INSDQualifier>
              <INSDQualifier_name>mol_type</INSDQualifier_name>
                                                                                      <INSDQualifier value>other
DNA</INSDQualifier_value>
                                                                  </INSDQualifier>
                                                                  <INSDQualifier id=“q2”>
              <INSDQualifier_name>organism</INSDQualifier name>
                                                                                      <INSDQualifier_value>synthetic
construct</INSDQualifier_value>
                                                                  </INSDQualifier>
                                                        </INSDFeature_quals>
                                              </INSDFeature>
                                    </INSDSeq_feature-table>
              <INSDSeq_sequence>atggattttaatgcaatcgctaaggtaacgcaggattatgaggaagcctgtgatcaaaaatcag
tattggtcgactatctattaaaactgatcgatagagctaatggcaatttaggcattgaacgggttaaacaagaggccataaaaatcatggattgtg
agttagccttccagcatcgacctgtgtttaatccccaaagcgatgattgctattgtgcattggatgtggtttttacaaagaacaaagatgtagatat
catcattaacacattttatgtacataaatatggctttgtttttatcaaaaacgctgatgtatatgaaaactggttaactgatagagaaatggattta
ttaacagatgcgacgctaagagacaaagttttatcccctatagtgtcagcgctacttaaacacttagaaaactaa</INSDSeq_sequence>
                          </INSDSeq>
              </SequenceData>
              <SequenceData sequenceIDNumber=“2”>
                          <INSDSeq>
                                    <INSDSeq_length>142</INSDSeq_length>
                                    <INSDSeq_moltype>AA</INSDSeq_moltype>
                                    <INSDSeq_division>PAT</INSDSeq_division>
                                    <INSDSeq_feature-table>
                                              <INSDFeature>
                                                        <INSDFeature_key>source</INSDFeature_key>
                                                        <INSDFeature_location>1 . . . 142</INSDFeature_location>
                                                        <INSDFeature_quals>
                                                                  <INSDQualifier>
              <INSDQualifier_name>mol_type</INSDQualifier_name>
              <INSDQualifier_value>protein</INSDQualifier_value>
                                                                  </INSDQualifier>
                                                                  <INSDQualifier id=“q4”>
              <INSDQualifier_name>organism</INSDQualifier_name>
                                                                                      <INSDQualifier_value>synthetic
construct</INSDQualifier_value>
                                                                  </INSDQualifier>
                                                        </INSDFeature_quals>
                                              </INSDFeature>
                                    </INSDSeq_feature-table>
              <INSDSeq_sequence>MDFNAIAKVTQDYEEACDQKSVLVDYLLKLIDRANGNLGI
ERVKQEAIKIMDCELAFQHRPVFNPQSDDCYCALDVVFTKNKDVDIIINTFYVHKYGFV
FIKNADVYENWLTDREMDLLTDATLRDKVLSPIVSALLKHLEN</INSDSeq_sequence>
                          </INSDSeq>
              </SequenceData>
</ST26SequenceListing>
```

What is claimed is:

1. A method of an application of Shewanella in a preparation of a nickase.

2. The method of the application according to claim 1, wherein the Shewanella is Shewanella oneidensis MR-1 (ATCC 700550).

3. A nickase derived from Shewanella oneidensis MR-1 (ATCC 700550).

4. The nickase according to claim 3, wherein the amino acid sequence is as shown in SEQ ID NO: 2.

5. A method of an application of the nickase according to claim 3 in a DNA single-strand break.

6. A nucleic acid encoding the nickase according to claim 3.

7. The nucleic acid according to claim 6, wherein the nucleotide sequence is as shown in SEQ ID NO: 1.

8. A method for preparing a nickase, comprising cultivating Shewanella to obtain a culture containing the nickase; wherein
the Shewanella is Shewanella oneidensis MR-1 (ATCC 700550).

9. A preparation, comprising at least one of the nickase according to claim 3 or a culture containing the nickase obtained by cultivating Shewanella oneidensis MR-1 (ATCC 700550), and auxiliary materials.

10. A method for a DNA single-strand break, wherein DNA is cut using the nickase according to claim 3 or a preparation, wherein the preparation comprises at least one of the nickase or a culture containing the nickase obtained by cultivating Shewanella oneidensis MR-1 (ATCC 700550), and auxiliary materials.

11. The method of the application according to claim 5, wherein the amino acid sequence is as shown in SEQ ID NO: 2.

12. The nucleic acid according to claim 6, wherein the amino acid sequence is as shown in SEQ ID NO: 2.

13. The nucleic acid according to claim 12, wherein the nucleotide sequence is as shown in SEQ ID NO: 1.

14. The preparation according to claim 9, wherein the amino acid sequence is as shown in SEQ ID NO: 2.

15. The method according to claim 10, wherein the amino acid sequence is as shown in SEQ ID NO: 2.


SEQUENCE LISTING (2 sequences)

SEQ ID NO: 1 - DNA, 429 bp, synthetic construct
  misc_feature 1..429: note = The sequence is synthetized.
  source 1..429: mol_type = other DNA
atggattttaatgcaatcgctaaggtaacgcaggattatgaggaagcctgtgatcaaaaatcagtattggtcgactatctattaaaactgatcgatagagctaatggcaatttaggcattgaacgggttaaacaagaggccataaaaatcatggattgtgagttagccttccagcatcgacctgtgtttaatccccaaagcgatgattgctattgtgcattggatgtggtttttacaaagaacaaagatgtagatatcatcattaacacattttatgtacataaatatggctttgtttttatcaaaaacgctgatgtatatgaaaactggttaactgatagagaaatggatttattaacagatgcgacgctaagagacaaagttttatcccctatagtgtcagcgctacttaaacacttagaaaactaa

SEQ ID NO: 2 - protein, 142 aa, synthetic construct
  REGION 1..142: note = The sequence is synthetized.
MDFNAIAKVTQDYEEACDQKSVLVDYLLKLIDRANGNLGIERVKQEAIKIMDCELAFQHRPVFNPQSDDCYCALDVVFTKNKDVDIIINTFYVHKYGFVFIKNADVYENWLTDREMDLLTDATLRDKVLSPIVSALLKHLEN